(19) United States
(12) Patent Application Publication
(10) Pub. No.: US 2006/0173434 A1
(43) Pub. Date: Aug. 3, 2006

(54) Ultra thin absorbent article including a hot melt superabsorbent polymer composition

(76) Inventors: Paula K. Zoromski, St. Paul, MN (US); Fouad D. Mehawej, Forest Lake, MN (US); Sharf U. Ahmed, Woodbury, MN (US); Steven A. Jorissen, Vadnais Heights, MN (US); Eugene R. Simmons, Vadnais Heights, MN (US)

Correspondence Address: Kirsten K. Stone, 1200 Willow Lake Blvd., P.O. Box 64683, St. Paul, MN, 55164-0683, US

(21) Appl. No.: 11/050,490

(22) Filed: Feb. 2, 2005

(51) Int. Cl. A61F 13/15

(52) U.S. Cl. 604/374

(57) ABSTRACT

An absorbent article such as a panty liner that includes a liquid pervious top sheet, a first interior component, a liquid impervious back sheet having an exterior surface and an interior surface, a hot melt superabsorbent polymer composition disposed between the top sheet and the interior surface of the back sheet, the panty liner having a thickness no greater than 1 millimeter and exhibiting at least one of a 0.9% saline wet back no greater than 30% and a synthetic blood wet back of no greater than 40%.

### BACKGROUND OF THE INVENTION

The invention relates to ultra thin absorbent articles that include a hot melt superabsorbent polymer composition.

A quest in the manufacture of absorbent articles for personal hygiene, such as panty shields, sanitary napkins and diapers, is to make the article as thin as possible while maintaining suitable functional properties such as liquid absorption capacity, wet back, penetration time, and penetration rate.

In the past, panty shields, sanitary napkins and diapers were constructed to include an absorbent core made from cellulosic fibers (e.g., air laid pulp), synthetic fibers and combinations thereof. The synthetic fibers were often provided in the form of a nonwoven web and cellulosic fibers were often provided in the form of a loose fluff of fibers. These cores were bulky and added thickness to the articles in which they were incorporated.

Attempts to increase the absorbent capacity or rate of absorption of an absorbent article have often resulted in an increase in the amount of absorbent material, such as cellulosic fluff and absorbent fibers, present in the absorbent article. The increased amount of material resulted in increased bulk of the absorbent article.

The development of superabsorbent polymers provided a mechanism for increasing absorbent capacity. Superabsorbent polymer in the form of particles, for example, has been added to cellulosic fiber and synthetic fiber cores to increase the absorption capacity of the core. Superabsorbent polymer particles form a dry powder, which is difficult to maintain in a desired location. In addition, superabsorbent polymer particles can tear thin films of polyethylene, which is undesirable since polyethylene films are often used as the barrier in many absorbent articles.

Hot melt superabsorbent polymer compositions have been developed in which the superabsorbent polymer particles are provided in a hot melt matrix. Hot melt superabsorbent polymer compositions have been utilized in conjunction with air laid cores.

Panty liners, which are generally designed for absorbing fluids (e.g., body exudates such as menses, sweat, blood, mucous, and urine) protecting clothing or both, are typically constructed to include an airlaid nonwoven top sheet, a polyethylene film back sheet, and an absorbent core disposed between the airlaid nonwoven and the polyethylene film. Active lifestyles and the myriad of exercise and physical activities in which people participate fuel the need for an ever thinner and more elastic panty liner while maintaining the performance properties thereof.

### SUMMARY

In one aspect, the invention features a panty liner that includes a liquid pervious top sheet, a first interior component, a liquid impervious back sheet, and, a hot melt superabsorbent polymer composition disposed between the top sheet and the back sheet, the panty liner having a thickness no greater than 1 millimeter and exhibiting at least one of a 0.9% saline wet back no greater than 30% and a synthetic blood wet back of no greater than 40%.

In one embodiment, the panty liner has a thickness no greater than 0.7 millimeter. In the other embodiment, the panty liner has a thickness no greater than 0.5 millimeter. In some embodiments, the panty liner has a thickness no greater than 0.3 millimeter. In another embodiment, the thermoplastic superabsorbent polymer composition is adhered to at least one of the top sheet and the back sheet.

In one embodiment, the panty liner exhibits a 0.9% saline wet back no greater than 20%. In some embodiments, the panty liner exhibits a 0.9% saline wet back of no greater than 10%.

In other embodiments, the panty liner exhibits a synthetic blood wet back of no greater than 35%.

In another embodiment, the panty liner exhibits a 0.9% saline penetration time of no greater than 10 seconds. In some embodiments, the panty liner exhibits a synthetic blood penetration time of no greater than 25 seconds.

In one embodiment, the first interior component includes a gel barrier. In some embodiments, the first interior component includes tissue. In other embodiments, the top sheet includes a biodegradable nonwoven web, the back sheet includes a biodegradable polymer film, and the first interior component includes cellulose tissue.

In another embodiment, the hot melt superabsorbent polymer composition is present in the panty liner in an amount no greater than 0.5 gram.

In some embodiments, the panty liner further includes a positioning adhesive adhered to the exterior surface of the back sheet, and optionally a release liner disposed on the positioning adhesive.

In one embodiment, the panty liner consists essentially of a liquid pervious top sheet, a first interior component, a liquid impervious back sheet that includes an exterior surface and an interior surface, a hot melt superabsorbent polymer composition disposed between the top sheet and the interior surface of the back sheet, optionally adhesive composition disposed between the top sheet and the back sheet, optionally a positioning adhesive adhered to the exterior surface of the back sheet, and optionally a release liner.

In another embodiment, the adhesive composition is present, the first component being bonded to the top sheet through the adhesive composition and the hot melt superabsorbent polymer composition being bonded to at least one of the first component and the back sheet through the adhesive composition.

In other embodiments, the panty liner includes a liquid pervious top sheet, a first interior component, a liquid impervious back sheet, and a hot melt superabsorbent polymer composition disposed between the top sheet and the back sheet, the panty liner having a thickness no greater than 1 millimeter (mm).

In one aspect, the invention features an absorbent article that includes a liquid pervious top sheet, a liquid impervious back sheet, and a hot melt superabsorbent polymer composition disposed between the top sheet and the back sheet, the absorbent article having a thickness no greater than 1 millimeter exhibiting at least one of a 0.9% saline wet back no greater than 30% and a synthetic blood wet back of no greater than 40%.

The invention features ultra thin absorbent articles such as panty liners that include, e.g., a top sheet, a first interior component, a back sheet and a hot melt superabsorbent polymer composition disposed between the top sheet and the back sheet. The invention also features an ultra thin panty liner that includes an ultra thin absorbent core that includes hot melt superabsorbent polymer composition. The hot melt superabsorbent polymer composition can also provide the absorbent function of a traditional absorbent core in absorbent articles.

The disclosed construction enables the panty liner to be constructed without a bulky absorbent core. In particular, the panty liner can be constructed to be free of a cellulosic core or a nonwoven core. By eliminating the need for a core other than a thin coating(s) of hot melt superabsorbent polymer (e.g., by eliminating a nonwoven web or cellulosic core), the panty liner can be constructed to be extremely thin including, e.g., having a thickness no greater than 1 mm or even no greater than 0.5 mm.

The invention also features an ultra thin panty liner that exhibits good (e.g., low levels of) wet back, or even is free of wet back, and good liquid penetration time.

### GLOSSARY

In reference to the invention, these terms have the meanings set forth below:

As used herein, the term “panty liner” refers to an article that is worn by females in an undergarment adjacent to the pudendal region and that is intended to absorb and contain the various exudates that are discharged from the body (e.g., blood, menses, urine, sweat, and mucous).

The term “consisting essentially of” means that to the extent an article includes additional components, the additional components are not absorbent.

Other features and advantages will be apparent from the following description of the preferred embodiments, from the claims and from the accompanying drawings wherein like characters of reference designate corresponding parts throughout the several views and in which:

### BRIEF DESCRIPTION OF THE DRAWINGS

FIG. 1 is a plan view of a body facing side of a panty liner according to one embodiment of the invention;

FIG. 2 is a plan view of a garment facing side of the panty liner of FIG. 1 with the adhesive and release liner removed;

FIG. 3 is a cross-sectional view of the panty liner of FIG. 1, taken along the line A-A;

FIG. 4 is a plan view of a body facing side of a panty liner with the top sheet removed according to another embodiment;

FIG. 5 is a plan view of a body facing side of a panty liner with the top sheet removed according to another embodiment;

FIG. 6 is a plan view of a body facing side of a panty liner with the top sheet removed according to another embodiment;

FIG. 7 is a plan view of a body facing side of a panty liner with the top sheet removed according to another embodiment;

FIG. 8 is a plan view of a body facing side of a panty liner with the top sheet removed according to another embodiment;

FIG. 9 is a plan view of a body facing side of a panty liner with the top sheet removed according to another embodiment;

FIG. 10 is a plan view of a body facing side of a panty liner with the top sheet removed according to another embodiment;

FIG. 11 is a plan view of a body facing side of a panty liner with the top sheet removed according to another embodiment;

FIG. 12 is a plan view of a body facing side of a panty liner with the top sheet removed according to another embodiment;

FIG. 13 is a plan view of a body facing side of a panty liner with the top sheet removed according to another embodiment.

### DETAILED DESCRIPTION

The ultra thin absorbent article includes a liquid pervious top sheet, a liquid impervious back sheet, and hot melt superabsorbent polymer composition disposed between the top sheet and the back sheet. The article also preferably includes a first interior component disposed between the top sheet and the hot melt superabsorbent polymer composition, optionally a construction adhesive, optionally a positioning adhesive adhered to the exterior surface of the back sheet, and optionally a release liner adhered to the positioning adhesive.

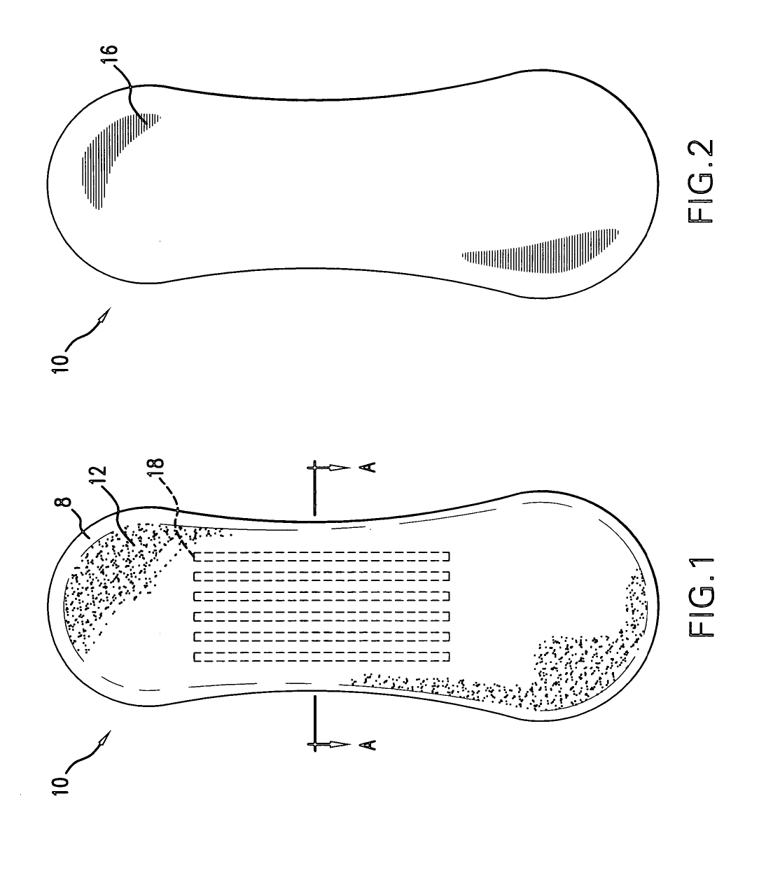

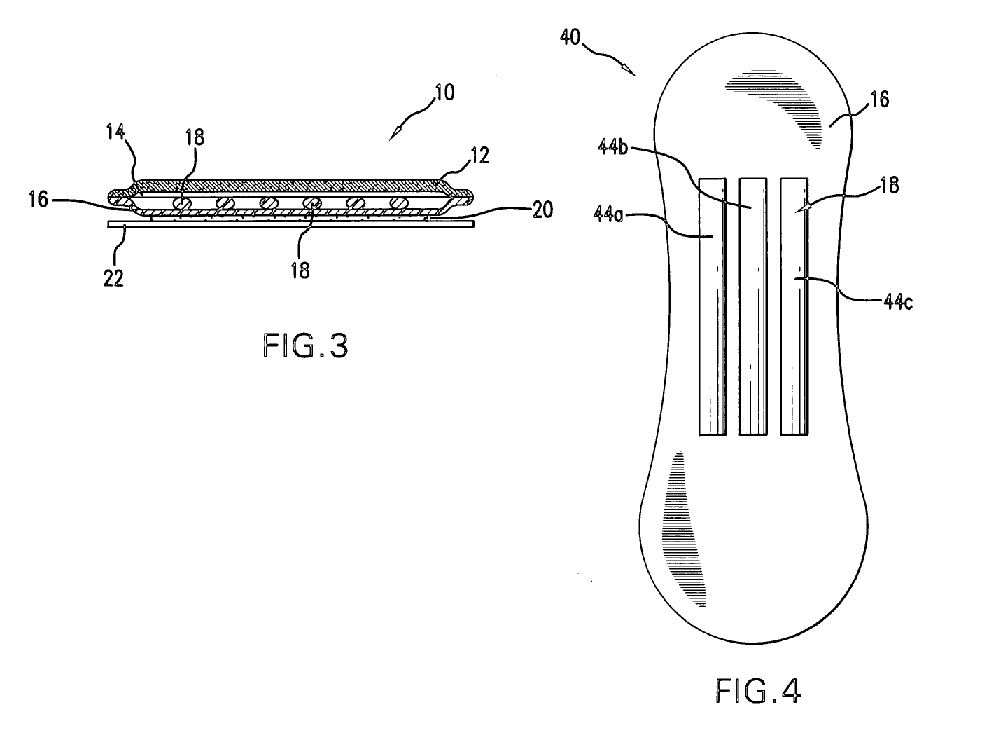

FIGS. 1-3 illustrate one embodiment of an ultra thin absorbent article in the form of a panty liner 10 that includes a liquid pervious top sheet 12, a first interior component 14, a liquid impervious back sheet 16, stripes of hot melt superabsorbent polymer composition 18 disposed between the first interior component 14 and the back sheet 16, positioning adhesive 20 adhered to the exterior surface of the back sheet 16, and a release liner 22 adhered to the positioning adhesive 20. The release liner 22 can be removed to expose the positioning adhesive 20 thereby making it available for contact with and adhesion to a substrate, e.g., a garment of the wearer. A seam 8, which affixes the components in position within the panty liner 10, extends along the periphery of the panty liner 10.

The panty liner is ultra thin having a thickness of, e.g., no greater than 1 mm, no greater than about 0.7 mm, no greater than about 0.5 mm, no greater than about 0.4 mm, no greater than about 0.3 mm, or even no greater than about 0.2 mm in thickness.

The panty liner preferably exhibits at least one of a synthetic blood wet back no greater than 40%, no greater than 35%, no greater than 30%, no greater than 25%, or even no greater than 20%, and a 0.9% saline wet back of no greater than 25% wet back, no greater than 20% wet back, no greater than 15% wet back, no greater than 10% wet back or even no greater than 5% wet back.

The panty liner also preferably exhibits at least one of a synthetic blood penetration time of less than 35 seconds, or even less than 30 seconds, and a 0.9% saline penetration time of no greater than 15 seconds, no greater than 10 seconds, or even no greater than 5 seconds.

The hot melt superabsorbent polymer composition 18 present in the panty liner shown in FIGS. 1-3 is in the form of six thin stripes 18a-f extending along the mid section of the longitudinal extent of the panty liner 10 and spaced an equal distance apart from one another. Spacing the regions of hot melt superabsorbent polymer apart from one another allows the superabsorbent polymer to expand as it absorbs liquid. The stripes 18a-f are visible through the nonwoven top sheet and the first absorbent component due in part to a coloring agent present in the hot melt adhesive composition.

The configuration, location and amount of the hot melt hot melt superabsorbent polymer composition on the panty liner preferably optimizes the absorbent properties of the panty liner including, e.g., penetration time, penetration rate, wet back, and combinations thereof. The stripe(s) of hot melt superabsorbent polymer composition can exists in a variety of configurations including, e.g., single or multiple stripes (e.g., one, two, three, four, and five), and continuous or discontinuous stripes. Where multiple stripes of hot melt superabsorbent polymer are present any suitable spacing between the individual regions can exist including, e.g., constant (e.g., equal spacing distances) or variable spacing distances. The stripes can also be positioned in any orientation including, e.g., along the length of the panty liner, the width of the panty liner, at any angle to the longitudinal axis of the panty liner, at any angle to the latitudinal axis of the panty liner, and combinations thereof. The width and length of the stripes can also be constant or variable. The spacing between the regions of hot melt superabsorbent polymer composition is preferably sufficient to allow the superabsorbent polymer freedom to expand as it would when contacted with water.

Any amount of hot melt superabsorbent polymer composition can be present in the panty liner. Preferably the total weight of the hot melt superabsorbent polymer composition present in the panty liner is at least about 0.025 gram (g), at least about 0.05 g, at least about 0.08 g, no greater than about 1.5 g, no greater than about 1 g, no greater than about 0.5 g, no greater than about 0.25 g, no greater than about 0.15 g, or even about 0.1 g. The hot melt superabsorbent polymer composition can also be applied at a very thin coating weight to assist in minimizing the overall thickness of the panty liner. Any suitable coat weight can be used. Useful coat weights include, e.g., no greater than about 200 g/m², no greater than about 150 g/m², no greater than about 100 g/m², no greater than about 75 g/m², no greater than about 55 g/m², or even no greater than about 40 g/m², and combinations thereof.

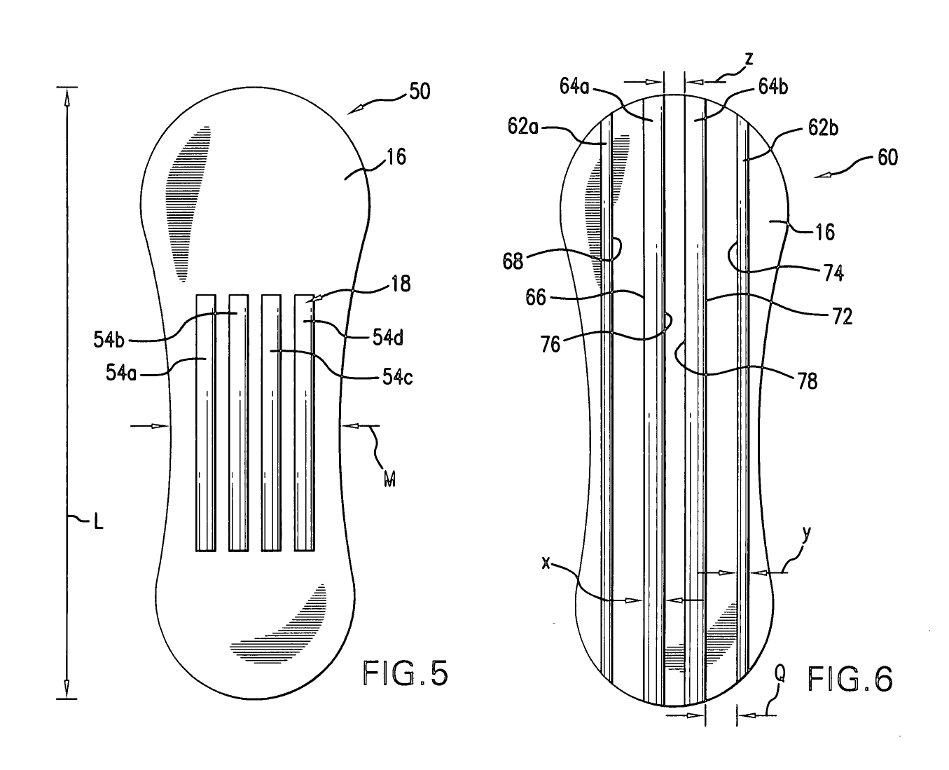

FIGS. 4 and 5 illustrate embodiments of a panty liner (40, 50) that include three stripes 44a-c and four stripes 55a-d of hot melt superabsorbent polymer composition 18, respectively.

FIG. 6 illustrates an embodiment of a panty liner 60 in which the hot melt superabsorbent polymer composition 18 is in the form of four stripes 62a, 62b, 64a, 64b having differing widths is shown in FIG. 6. The two interior stripes 64a, 64b have a first width X and the exterior stripes 62a, 62b have a second width Y that is smaller than the first width X. The distance Z between the interior edges 76, 78 of the interior stripes 64a, 64b, respectively, is less than the distance Q between the outer edges 66, 72 of the interior stripes 64a, 64b and the interior edges 68, 74 of the exterior stripes 62a, 62b. The stripes 62a, 62b, 64a, 64b extend the entire length of the longitudinal extent of the panty liner 60.

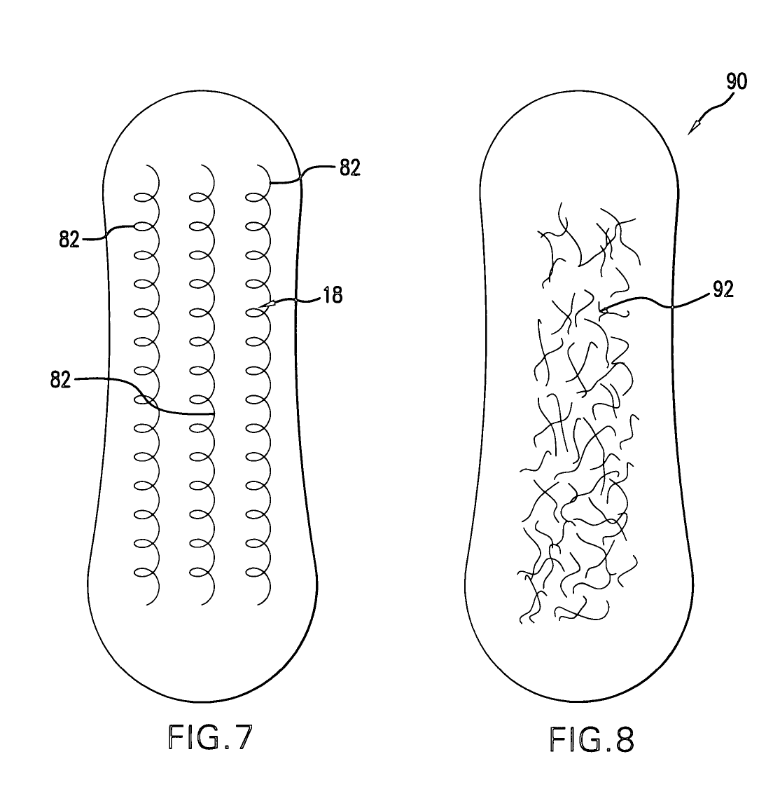

In FIG. 7, an embodiment of the panty liner 80 is shown in which the hot melt superabsorbent polymer composition 18 is in the form of three parallel spiral spray lines 82 applied using a spiral spray technique.

In FIG. 8 an embodiment of the panty liner 90 is shown in which the hot melt superabsorbent polymer composition 18 is in the form of a random spray pattern 92.

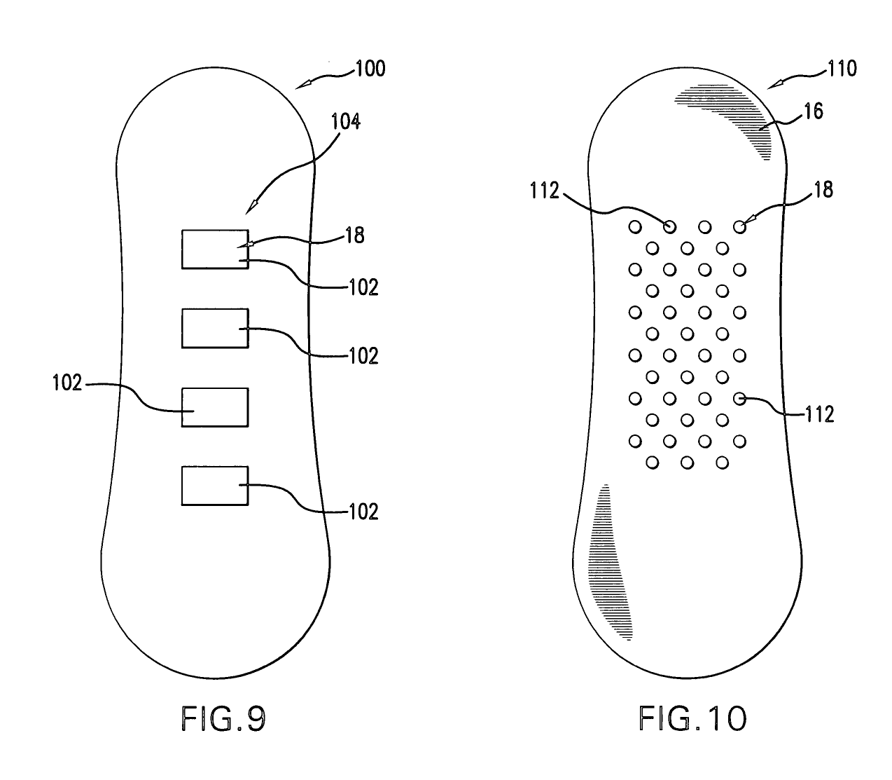

FIG. 9 illustrates an embodiment of a panty liner 100 that includes hot melt superabsorbent polymer composition 18 in the form of four rectangular blocks 102 positioned in a column 104 along the longitudinal extent of the panty liner 100.

In other embodiments, the hot melt superabsorbent polymer composition is present in a variety of other configurations including, e.g., a continuous film extending across at least a portion of the width and at least a portion of the length of the absorbent article, a pattern, and randomly, and in a variety of other shapes including, e.g., waves, dots, triangles, squares, rectangles, diamonds, pentagons, hexagons, circles, ellipsis, ovals, crosses, x's, flowers, leaves, petals, stars, and combinations thereof. FIG. 10 illustrates an embodiment of a panty liner 110 in which the hot melt superabsorbent polymer 18 is in a pattern of circular dots 112.

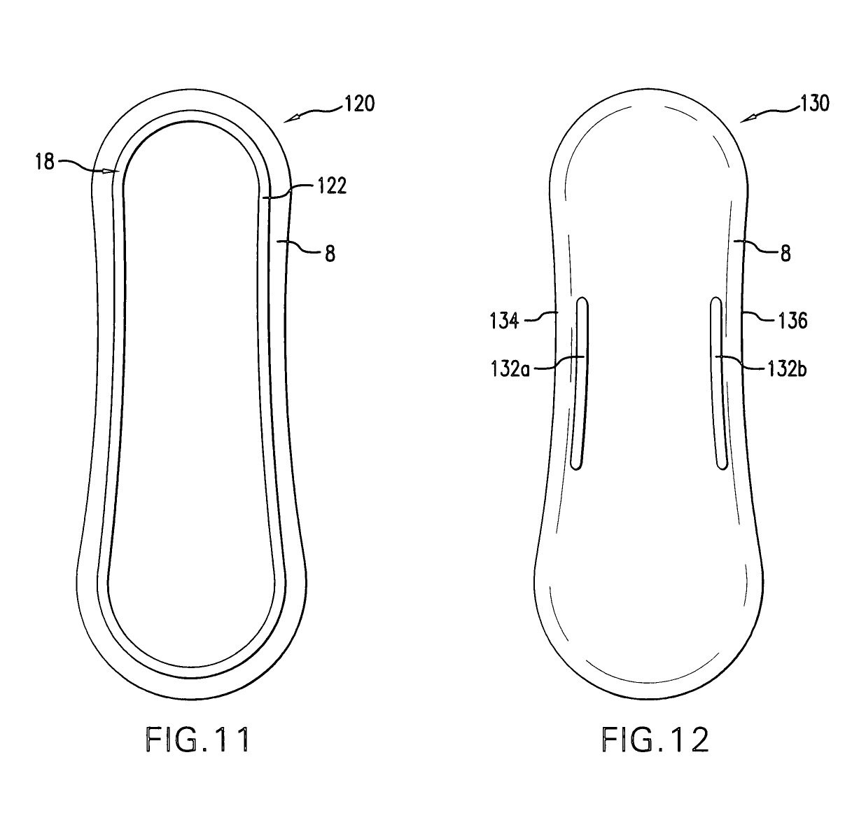

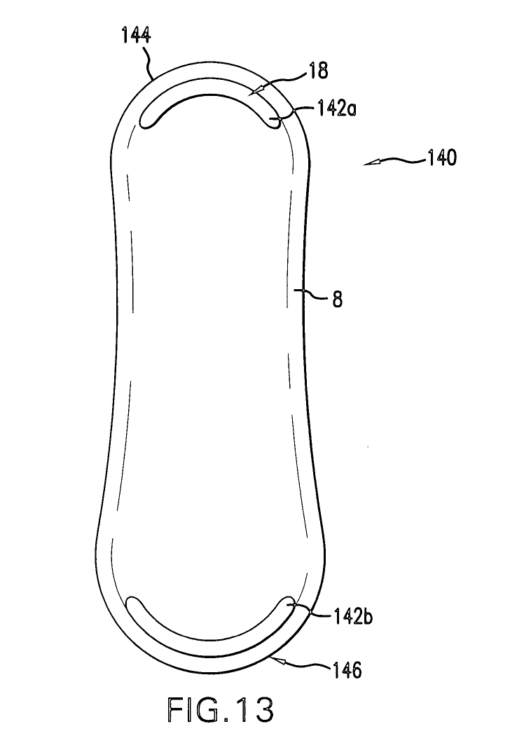

In other embodiments, the hot melt superabsorbent polymer is alternatively or additionally positioned near the periphery of the panty liner to prevent leakage, i.e., the movement of absorbed liquid out of the panty liner through the panty liner sides, seams or both. In FIG. 11, the panty liner 120 includes a continuous band 122 of hot melt superabsorbent polymer composition 18 extending along the periphery 124 of the panty liner 120 and within the area defined by the peripheral seam 8. In another embodiment, elongated regions 132a, 132b of hot melt superabsorbent polymer composition 18 are positioned along the lateral sides 134, 136 of the panty liner 130 as shown in FIG. 13. Alternatively or in addition, the panty liner 140 includes hot melt superabsorbent polymer composition 18 in the form of elongated regions 142a, 142b located at the ends 144, 146, respectively, of the longitudinal extent of the panty liner 140 as shown in FIG. 13.

The hot melt superabsorbent polymer composition can be applied to any component of the construction including, e.g., the back sheet, the first absorbent layer, or a combination thereof, in a variety of forms including, e.g., a coating (e.g., a continuous or discontinuous coating), a film (e.g., a continuous or discontinuous film), and a spray pattern, using any suitable technique including, e.g., contact coating, noncontact coating, spraying (e.g., spiral spraying and random spraying), extrusion, slot coating, melt blown, foaming, engraved roller, gravure, and screen printing. Preferably the hot melt superabsorbent polymer composition is adhered to the back sheet through an adhesive composition, e.g., a pressure sensitive adhesive composition.

The hot melt superabsorbent polymer composition can be any suitable hot melt superabsorbent polymer composition that includes a thermoplastic polymer and superabsorbent polymer including, e.g., a hot melt adhesive composition that includes superabsorbent polymer. Useful thermoplastic polymers include, e.g., styrenic block copolymers, polyolefins (e.g., amorphous and crystalline polyolefins including homogeneous and substantially linear ethylene/alpha-olefin interpolymers), interpolymers and copolymers of ethylene including, e.g., ethylene-vinyl acetate, ethylene-vinyl acetate ethylene-acrylic acid, ethylene-methacrylic acid, ethylene-methyl acrylate, ethylene-ethyl acrylate and ethylene n-butyl acrylate and derivatives (e.g., incorporating at least two comonomers), polyacrylic acids, polymethacrylic acids, polyacrylates, polyvinyl acetates, polylactic acids, polylactides, caprolactone polymers, poly (hydroxy-butyrate/hydroxyvalerate), polyvinyl alcohols, polyesters, copolyesters (e.g., biodegradable copolyesters), poly(ethylene oxide)polyether amide, polyester ether block copolymers, polyvinyl pyrrolidone, polyvinyl pyrrolidone-vinyl acetate copolymer, polyetheroxazoline, polyvinyl ethers (e.g., polyvinyl methyl ether), polyamides, polyacrylamide, and combinations thereof.

A wide variety of block copolymers are useful including, e.g., A-B-A triblock copolymers, A-B diblock copolymers, and (A-B)n radial block copolymers, and branched and grafted versions thereof, wherein the A blocks are non elastomeric polymer blocks, typically comprising polystyrene, and the B blocks are unsaturated conjugated diene or hydrogenated version thereof. Suitable B blocks include, e.g., isoprene, butadiene, ethylene/butylene (hydrogenated butadiene), ethylene/propylene (hydrogenated isoprene), and combinations thereof. Useful block copolymers are commercially available under the KRATON D and G series of trade designations from Shell Chemical Company (Houston, Tex.), EUROPRENE Sol T trade designation from EniChem (Houston, Tex.), and Vector® series of trade designations from Exxon (Dexco) (Houston, Tex.).

Useful commercially available polyolefins include, e.g., AFFINITY substantially linear ethylene polymers polyolefin plastomers from The Dow Chemical Company (Midland, Mich.) and EXACT homogeneous linear ethylene polymers from Exxon Chemical Company (Houston, Tex.). Useful amorphous polyolefins and amorphous polyalphaolefins include homopolymers, copolymers, and terpolymers of C₂-C₈ alphaolefins. Useful commercially available amorphous polyalphaolefins include, e.g., REXTAC and REXFLEX propylene based homopolymers, ethylene-propylene copolymers and butene-propylene copolymers available from Rexene (Dallas, Tex.), VESTOPLAST alpha-olefin copolymers available from Hüls (Piscataway, N.J.).

Any suitable superabsorbent polymer can be included in the composition. Superabsorbent polymers are also referred to as water-insoluble absorbent hydrogel-forming polymers, “hydrogel-forming” polymers, and “hydrocolloids.” Superabsorbent polymers are able to absorb many times their own weight in water. Useful superabsorbent polymers include at least partially crosslinked, at least partially neutralized polymers that gel when contacted with water and are preferably substantially water insoluble. Suitable superabsorbent polymers include, e.g., polysaccharides (e.g., carboxymethyl starch, carboxymethyl cellulose, and hydroxypropyl cellulose), polyvinyl alcohol, polyvinyl ethers, polyvinyl pyridine, polyvinyl morpholinione, N,N-dimethylaminoethyl, N,N-diethylaminopropyl, acrylates, methacrylates, and the quaternary salts thereof. The superabsorbent polymer preferably includes a plurality of functional groups, e.g., sulfonic acid groups, carboxy groups and combinations thereof.

Suitable superabsorbent polymers are prepared from polymerizable, unsaturated, acid-containing monomers including, e.g., olefinically unsaturated acids and anhydrides having at least one carbon-carbon olefinic double bond including, e.g., olefinically unsaturated carboxylic acids and acid anhydrides, olefinically unsaturated sulfonic acids and combinations thereof. Useful olefinically unsaturated carboxylic acid and carboxylic acid anhydride monomers include, e.g., acrylic acid, methacrylic acid, ethacrylic acid, chloroacrylic acid, cyanoacrylic acid, crotonic acid, phenylacrylic acid, acrytoxypropionic acid, sorbic acid, chlorosorbic acid, angelic acid, cinnamic acid, p-chlorocinnamic acid, stearylacrylic acid, itaconic acid, citraconic acid, mesaconic acid, glutaconic acid, aconitic acid, maleic acid, fumaric acid, tricarboxyethylene acid anhydride, maleic acid anhydride, and combinations thereof.

Useful olefinically unsaturated sulfonic acid monomers include aliphatic and aromatic vinyl sulfonic acids (e.g., vinylsulfonic acid, allyl sulfonic acid, vinyl toluene sulfonic acid and styrene sulfonic acid), acrylic and methacrylic sulfonic acids (e.g., sulfoethyl acrylate, sulfoethyl methacrylate, sulfopropyl acrylate, sulfopropyl methacrylate, 2-hydroxy-3-methacryloxypropyl sulfonic acid and 2-acrylamide-2-methylpropane sulfonic acid), and combinations thereof.

Useful superabsorbent polymers that include carboxy groups include, e.g., hydrolyzed starch-acrylonitrile graft copolymers, partially neutralized hydrolyzed starch-acrylonitrile graft copolymers, starch-acrylic acid graft copolymers, partially neutralized starch-acrylic acid graft copolymers, saponified vinyl acetate-acrylic ester copolymers, hydrolyzed acrylonitrile or acrylamide copolymers, slightly network crosslinked polymers of any of the foregoing copolymers, partially neutralized polyacrylic acid, and slightly network crosslinked polymers of partially neutralized polyacrylic acid. These polymers are disclosed, e.g., in U.S. Pat. Nos. 4,076,663, 4,093,776, 4,666,983 and 4,734,478 and incorporated herein.

The absorbent gelling particles used in the present invention can have a size, shape, morphology and combinations thereof. The superabsorbent particles preferably are spherical and preferably have an average particle size no greater than about 400 μm, no greater than about 350 μm, no greater than about 200 μm, no greater than about 150 μm, no greater than about 100 μm, no greater than about 100 μm, no greater than about 50 μm, no greater than about 40 μm, at least about 10 μm, or even about 20 μm to about 30 μm.

Useful commercially available superabsorbent particles include, e.g., sodium polyacrylate superabsorbent particles available under the AQUA KEEP series of trade designations including, e.g., particles having a median particle size of from about 20 μm to about 30 μm available under the trade designation AQUA KEEP 10SH-NF, particles having an average particle size of from 200 μm to 300 μm available under the trade designation AQUA KEEP 10SH-P, particles having an average particle size of from 320 μm to 370 μm available under the trade designation AQUA KEEP SA60S, particles having an average particle size of from 350 μm to 390 μm available under the trade designations AQUA KEEP SA60SX, SA55SX II and SA 60SL II, and particles having an average particle size of from 250 μm to 350 μm available under the trade designation AQUA KEEP SA60N TYPE II from Sumitomo Seika Chemicals Col, Ltd. (Japan). Useful superabsorbent polymer forms include, e.g., particles, granules, flakes, pulveruients, interparticle aggregates, interparticle crosslinked aggregates, fibers, foams, and combinations thereof.

The hot melt superabsorbent polymer composition can also include other additives including, e.g., plasticizers, tackifiers, waxes, antioxidants, pigments, fillers (e.g., hydrophilic fillers), surfactants and combinations thereof.

Useful hot melt superabsorbent polymer compositions are described in, e.g., U.S. Pat. Nos. 6,534,572, and 6,458,877, and U.S. patent application Ser. Nos. 10/050,375 (published as U.S. Publication No. 2003/0134552), and 11/007,470, and incorporated herein. Useful hot melt superabsorbent polymer compositions are commercially available under the HYDROLOCK trade designation from H.B. Fuller Company (Vadnais Heights, Minn.).

The top sheet 12 is a liquid pervious component (i.e., pervious to water-based liquids, e.g., body exudates including, e.g., menses, urine, water, sweat, blood, mucous, and combinations thereof) that permits water-based liquids to readily penetrate through its thickness. The top sheet 12 is preferably compliant, soft feeling, and non-irritating to the wearer's skin. The top sheet can also have elastic characteristics allowing it to stretch in at least one direction.

The panty liner can include any suitable top sheet. Suitable top sheets include, e.g., woven and nonwoven webs, films (e.g., apertured formed thermoplastic films, apertured plastic films, hydroformed thermoplastic films and reticulated thermoplastic films), foams (e.g., porous foams and reticulated foams), thermoplastic scrims, and combinations thereof. The top sheet can be made from a variety of polymers including, e.g., synthetic polymers, natural polymers and combinations thereof. Suitable polymers include, e.g., polyolefins (e.g., polyethylene, low density polyethylene, and linear low density polyethylene, polypropylene, and combinations thereof), polyacrylates, ethylene vinyl acetate, and copolymers and blends thereof. Suitable woven and nonwoven webs include, e.g., natural fibers (e.g., wood and cotton fibers), synthetic fibers (e.g., polymeric fibers including, e.g., polyester, polypropylene, and polyethylene fibers), biodegradable polymers (e.g., biodegradable polyester), and combinations thereof.

An example of a useful top sheet is one that is or includes an apertured formed film. Apertured formed films are pervious to body exudates and, if properly apertured, have a reduced tendency to allow liquids to pass back through and rewet the wearer's skin. Thus, the surface of the formed film that is in contact with the body remains dry, thereby reducing body soiling and creating a more comfortable feel for the wearer. Suitable formed films are described in, e.g., U.S. Pat. Nos. 3,929,135, 4,324,246, 4,342,314, 4,463,045, 4,637,819, 4,780,352, 5,006,394, and 6,180,052. Useful top sheets are commercially available from Ahlstrom (Windsor Lock, Conn.), and First Quality Nonwoven (Greatneck, N.Y.) and under the SOFTSKIN trade designation from BBA Nonwovens (Nashville, Tenn.).

Useful top sheets have a thickness from about 0.025 millimeters (mm) to about 1.0 mm.

Useful methods of making nonwoven webs top sheets include, e.g., air laid, wet laid, meltblown, spunbonded, carded, thermally bonded, air-through bonded, powder bonded, latex bonded, solvent bonded, spunlaced, and combinations thereof.

The body surface of the top sheet is preferably hydrophilic to allow liquids to transfer more readily through the top sheet, which diminishes the likelihood that menstrual fluid will flow off the top sheet rather than flowing into and being absorbed by the absorbent core. The body contacting surface of the top sheet can be made hydrophilic by treatment with a surfactant. Suitable methods of treating a top sheet with a surfactant are described in, e.g., U.S. Pat. No. 4,950,254. Alternately, surfactant can be incorporated into the polymer of the top sheet as described in, e.g., in PCT Publication No. WO93/09741.

The first interior component is a component located between the back sheet and the top sheet. The first interior component is preferably in the form of a layer (e.g., a sheet). The first interior component is preferably coextensive with the area of the hot melt superabsorbent polymer composition, coextensive with the top sheet, or a combination thereof. The first interior component is liquid permeable and preferably gel impermeable (e.g., a gel barrier). The first interior component inhibits, and preferably prevents, the gel formed when the hot melt superabsorbent polymer composition is contacted with water from passing through the top sheet and contacting the wearer. Preferably the top sheet is in contact with the first interior component to increase the rate at which liquid penetrates the top sheet. Suitable methods for maintaining the top sheet in contact with the first interior component includes, e.g., bonding the top sheet to the first interior component, entangling fibers of the first interior component with the top sheet, fusing the top sheet to the first interior component, e.g., creating a number of discrete individual fusion bonds, and combinations thereof.

The first interior component is preferably conformable and non-irritating to a user's skin. The first interior component can be extensible. Suitable types of first interior components include, e.g., tissues (e.g., cellulose fiber tissue and synthetic fiber tissue). The first interior component can be made from a variety of materials including, e.g., wet laid cellulose fibers, creped cellulose, comminuted wood pulp, modified crosslinked cellulose fibers, cotton (e.g., woven cotton), synthetic fibers and combinations thereof.

Other useful first interior components include, e.g., woven webs, nonwoven webs and combinations thereof. These webs can include a variety of fibers including, e.g., synthetic, partially synthetic, natural, and partially natural fibers. Suitable synthetic fibers include polyester, polypropylene, polyethylene, nylo, viscose rayon, cellulose acetate fibers and combinations thereof. Suitable natural fibers include, e.g., cotton, cellulose, other natural fibers and combinations thereof.

Useful tissues are commercially available under the trade designations 1-PLYTISSUE from Cityforest Corp. (Ladysmith, Wis.).

Useful methods of making nonwoven webs include, e.g., air laid, wet laid, meltblown, spunbonded, carded, thermally bonded, air-through bonded, powder bonded, latex bonded, solvent bonded, spunlaced, and combinations thereof.

The first interior component is very thin and preferably has a thickness no greater than about 1 mm, no greater than 0.5 mm, no greater than 0.1 mm, or even about 0.08 mm. Useful first interior components have a basis weight of no greater than about 45 g/m², no greater than about 40 g/m², at least about 5 g/m² or even at least about 10 g/m².

The back sheet prevents exudates, and liquids absorbed by the panty liner, from wetting substrates including e.g., clothing (e.g., undergarments) that contact the panty liner. The back sheet preferably is liquid impervious (e.g., impervious to water-based exudates including, e.g., blood, menses, urine, water, mucous, and other exudates) and flexible. The back sheet can also exhibit elastic properties such that it can stretch in at least one direction. The back sheet can be breathable or nonbreathable. The term “breathable” refers to the property of allowing vapors to transfer therethrough, while preventing liquids from transferring therethrough. Suitable breathable films include hydrophilic films including monolithic films. The back sheet can also be biodegradable.

Useful back sheets include, e.g., polymeric films (e.g., thermoplastic films of polymers including e.g., polyethylene, polypropylene, polyester and ethylene vinyl acetate), composites, e.g., film-coated woven and nonwoven webs, and combinations thereof.

A composite back sheet that includes a woven or nonwoven web and a continuous film bonded thereto can be prepared according to a variety of methods including those methods disclosed in, e.g., U.S. Pat. Nos. 6,583,332, 5,827,252, and 6,843,874 and incorporated herein.

Useful commercially available back sheet materials are available under the product number P18-1401 from The Clopay Corporation (Cincinnati, Ohio) and breathable, microporous, monolithic films available from Tredegar Film Products (Richmond, Va.).

The back sheet can be treated, e.g., by embossing or finishing, to achieve, e.g., an embossed surface, a matte finish, and combination thereof, to provide a more clothlike appearance. The back sheet can also be a laminate, e.g., a spunbond/meltblown/spunbond construction that is commercially available, for example, under the trade designations SMS and SMMS from Kimberly Clark (Neenah, Wis.), and those nonwoven/film laminates described in U.S. Pat. No. 5,007,906.

The back sheet can also be extensible as described above with respect to the top sheet. Useful elastomeric film back sheet materials are described, e.g., in U.S. Pat. No. 4,746,180, and extensible adhesive film available under the trade designation Formula 198-338 from Findley Adhesives Co. (Wauwatosa, Wis.).

The back sheet can also be made extensible by performing a mechanical operation including, e.g., pleating, corrugating, ring rolling, and selfing, an example of which is described in U.S. Pat. No. 5,518,801.

Useful back sheets have a thickness of at least about 0.002 mm, no greater than about 2 mm, no greater than about 1 mm, no greater than about 0.5 mm, no greater than about 0.1 mm, or even about 0.05 mm.

The panty liner can also include a construction adhesive disposed between the top sheet and the back sheet to bond at least two of the components of the panty liner together. Useful placement of the construction adhesive includes, e.g., as indicated above, between the hot melt superabsorbent polymer composition and the back sheet so as to bond the hot melt superabsorbent polymer composition to the back sheet, between the top sheet and the first interior component to bond the first interior component to the top sheet, between the top sheet and the back sheet to bond the top sheet and back sheet to each other, and combinations thereof. Where the construction adhesive has been applied to the back sheet to bond the hot melt superabsorbent polymer composition to the back sheet, the exposed construction adhesive can also function to bond the top sheet to the back sheet or the first interior component to the back sheet.

The construction adhesive can be any suitable adhesive. Useful construction adhesives include, e.g., hot melt adhesives, pressure sensitive adhesives, hot melt pressure sensitive adhesives, and blends thereof. One example of a suitable commercially available construction adhesive is HL-1713 construction adhesive from H.B. Fuller Company (Vadnais Heights, Minn.).

The exterior surface of the panty liner can include a positioning adhesive for attaching the panty liner to a substrate including, e.g., the clothing (e.g., undergarment) of a wearer. The positioning adhesive can be any suitable adhesive including, e.g., pressure sensitive adhesives. Useful positioning adhesives are described in, e.g., U.S. Pat. No. 4,917,697. Suitable commercially available positioning adhesives are commercially available, e.g., under the trade designations HL-2238ZP, HL-1461-XZP, and LUNATAC D-1053 from H.B. Fuller Co. (Vadnais Heights, Minn.).

Any suitable release liner can be used including, e.g., kraft paper, semi-bleached kraft paper, silicone treated kraft paper, polyethylene, polyethylene-coated paper, polypropylene, silicone-base release agent treated (e.g., coated) sheets (e.g., paper), fluorine-base release agent treated sheets, and combinations thereof. Other useful release liners include a water-sensitive film having a release coating that includes release polymers suitable examples of which include polyolefins, fluoropolymers, and silicones. Useful water sensitive films include, e.g., polyethylene oxide, ethylene oxide/propylene oxide copolymers, polymethacrylic acid, polymethacrylic acid copolymers, polyvinyl alcohol, poly(2-ethyl oxazoline), polyvinyl methyl ether, polyvinyl pyrrolidone/vinyl acetate copolymers, methyl cellulose, ethyl cellulose, hydroxypropyl cellulose, hydroxypropyl methyl cellulose, ethyl hydroxyethyl cellulose, methyl ether starch, poly (n-isopropyl acrylamide), poly N-vinyl caprolactam, polyvinyl methyl oxazolidone, poly (2-isopropyl-2-oxazoline), poly (2,4-dimethyl-6-triazinyl ethylene), polyvinyl alcohol and combinations thereof. Suitable release liners are also described in, e.g., U.S. Pat. Nos. 4,917,697 and 6,258,427 and incorporated herein.

Any commercially available release liners commonly used for such purposes can also be utilized herein including, e.g., BL30MG-A Silox 4P/0 release liner and AKROSIL high release paper available from Akrosil Corporation (Menasha, Wis.).

The components of the panty liner can be assembled in any suitable manner. In the embodiment shown in FIGS. 1-3, the components are assembled in a sandwich configuration with the components sized so that the edges of the top sheet and the back sheet extend outward beyond the edges of the first absorbent layer. The top sheet and the back sheet are preferably at least partially peripherally joined using known techniques. As shown in FIG. 1, the top sheet is preferably secured to the back sheet along a seam 8. The seam is preferably liquid impervious. The seam can be formed by any suitable means including, e.g., gluing, crimping, heat sealing, ultrasonic bonding, and combinations thereof.

The components of the panty liner can be joined together using any suitable mechanism including, e.g., adhesives, stitching, heat bonding, pressure bonding, dynamic mechanical bonding, ultrasonic bonding, intermingling of fibers or components of the panty liner, entanglement of fibers or components of the panty liner, simultaneously or sequentially extruding at least two components of the panty liner, and combinations thereof. Methods of attaching components of a panty liner to each other are well known to the skilled artisan.

The components can be joined together in any suitable manner including, e.g., joining such that each component is directly secured to another component, a component is secured to an intermediate component, a component is integral with another component, and combinations thereof.

The invention will now be described by way of the following examples.

### EXAMPLES

Test Procedures

Test procedures used in the examples include the following.

Thickness

The thickness is measured by placing the thickest part of the absorbent article in an Ames pocket thickness gauge and measuring the thickness. The thickness of the absorbent article is measured four times and the highest reading is reported as the thickness of the article.

Penetration Time Test Method

A panty liner is placed on a work bench. A Plexiglas plate, 10 cm×19.5 cm, having a centrally located hole 2.3 cm in diameter, is placed on the panty liner such that the centrally located hole of the Plexiglas plate is positioned over the middle of the panty liner. One milliliter of fluid is added to panty liner through the hole in the center of the Plexiglas using a pipette and a timer is simultaneously started. The timer is stopped when the liquid completely disappears form the surface of the panty liner. The time is recorded in seconds and deemed the Penetration Time.

Penetration Rate Test Method

The penetration rate is calculated by dividing the amount of liquid used in the Penetration Time Test by the Penetration time. The rate is reported in milliliters per second.

0.9% Saline Wet Back Test Method

The percent wet back is determined by subjecting a panty liner to the Penetration time test method using 0.9% saline solution, removing the Plexiglas from the panty liner, waiting for ten minutes, weighing 4 g of Whatman #1 or VWR #415 9.0 cm filter paper, placing the filter paper on the spot in the middle of the panty liner, placing the Plexiglas on top of the filter paper and then placing a 3.13 kg weight on the Plexiglas. After 30 seconds, the weight and the Plexiglas are removed. The filter paper is weighed. The amount of wet back is calculated as the weight of the wet filter paper minus the weight of the dry filter paper.

Synthetic Blood Wet Back Test Method

The synthetic blood wet back test method is conducted according to the 0.9% Saline Wet Back Test Method with the exception that synthetic blood (of the type or equivalent available from Johnson, Moen & Co., Rochester, Minn.) is used instead of 0.9% saline.

### Example 1

A panty liner having the general shape shown in FIG. 5, a thickness of 0.5 mm, a maximum longitudinal extent (L) of 14 cm, and a smallest latitudinal extent (M) at the middle of the panty liner of 5.1 cm, is constructed by hand to include a nonwoven spunbond polypropylene top sheet having a thickness of 0.12 mm, and basis weight of 15 g/m bonded to an ACCUWIPE wet laid cellulose tissue (Fort James Corp., Deerfield, Ill.) through HL-1713 construction adhesive (H.B. Fuller Company), and four stripes of HYDROLOCK hot melt superabsorbent polymer composition (H.B. Fuller Company) bonded to a polyethylene back sheet through the same construction adhesive. Each stripe of HYDROLOCK hot melt superabsorbent polymer composition has the dimensions 4 mm×100 mm, the stripes are parallel to each other spaced apart a distance of 4 mm, and have a coating weight of 40 g/m². The total amount of HYDROLOCK on the panty liner, as weighed, is 0.1 g.

### Example 2

A panty liner is constructed as described above in Example 1 with the exception that the coating weight of each stripe is 50 g/m² and the theoretical total amount of HYDROLOCK on the panty liner is 0.08 g.

### Example 3

A panty liner is constructed as described above in Example 1 with the exception that the coating weight of each stripe is 60 g/m² and the theoretical total amount of HYDROLOCK on the panty liner is 0.096 g.

### Example 4

A panty liner is constructed as described above in Example 1 with the exception that the coating weight of each stripe is 130 g/m² and the theoretical total amount of HYDROLOCK on the panty liner is 0.208 g.

Panty liners prepared according to Examples 1-4, when tested according to the Penetration Rate Test Method using 0.9% saline, the Penetration Rate Test Method using synthetic blood, 0.9% Saline Wet Back Test Method, and Synthetic Blood Wet Back Test Method, are expected to provide the results set forth below in Table 1 as averaged over a set of two test specimens for each example.

TABLE 1

| | HMSAP Stripe | Penetration | % Wet Back | Penetration | % Wet Back |
|---|---|---|---|---|---|
| | Coating Weight | Time (s) 0.9 | 0.9% | Time (s) | Synthetic |
| Sample | (g/m²) | % Saline | Saline | Synthetic Blood | Blood |
| 1A | 130 | 3.5 | 2.8 | NT | NT |
| 2A | 60 | 6.5 | 3.8 | NT | NT |
| 2B | 60 | NT | NT | 20.5 | 34.3 |
| 3A | 50 | 7 | 9.4 | NT | NT |
| 3B | 50 | NT | NT | 17 | 27.4 |
| 4A | 40 | 7.5 | 14.8 | NT | NT |
| 4B | 40 | NT | NT | 23.5 | 35.7 |

HMSAP = hot melt superabsorbent polymer composition
NT = not tested

### Example 5

A panty liner having a thickness of approximately 0.5 mm is constructed by hand to include a nonwoven spunbond polypropylene top sheet having a thickness of 0.12 mm and a basis weight of 15 g/m², bonded to an ACCUWIPE wet laid cellulose tissue through HL-1713 construction adhesive, and four stripes of HYDROLOCK hot melt superabsorbent polymer composition (H.B. Fuller Company) bonded to a polyethylene film back sheet through the same construction adhesive, as shown in FIG. 5. Each stripe of HYDROLOCK hot melt superabsorbent polymer composition has the dimensions 4 mm×100 mm, and a coating weight as set forth in Table 2 below. The stripes are parallel to each other and spaced apart a distance as indicated in Table 2 below. The total amount of HYDROLOCK on the panty liner, as weighed, is 0.1 g.

Panty liners prepared according to Example 5, when tested according to the Penetration Rate Test Method using 0.9% saline, the Penetration Rate Test Method using synthetic blood, the 0.9% Saline Wet Back Test Method, and the Synthetic Blood Wet Back Test Method, are expected to provide the results set forth below in Table 2.

TABLE 2

| | | | Penetration | Penetration | % Wet Back | % Wet Back |
|---|---|---|---|---|---|---|
| | Spacing | HMSAP | Time (s) | Time (s) | 0.9% | Synthetic |
| Example 5 | (mm) | (g) | 0.9% Saline | Synthetic Blood | Saline | Blood |
| A | 4 | 0.1131 | 11 | NT | 3.9 | NT |
| B | 4 | 0.0948 | 12 | NT | 5.1 | NT |
| C | 3 | 0.0935 | 12 | NT | 4.5 | NT |
| D | 3 | 0.1008 | 9 | NT | 6.3 | NT |
| E | 4 | 0.0911 | 36 | NT | 27.8 | NT |
| F | 3 | 0.0921 | 36 | NT | 26.7 | NT |
| G | 4 | 0.0928 | NT | 30 | NT | 29.5 |
| H | 4 | 0.0885 | NT | 28 | NT | 32.4 |
| I | 4 | 0.1022 | NT | 31 | NT | 29.2 |
| J | 4 | 0.0944 | NT | 28 | NT | 28.4 |
| K | 3 | 0.0913 | NT | 25 | NT | 30.1 |
| L | 3 | 0.0899 | NT | 32 | NT | 28.6 |
| M | 3 | 0.0920 | NT | 22 | NT | 28. |

NT = not tested

Other embodiments are within the claims. Although the absorbent article has been described with reference to a panty liner, the absorbent article can be any absorbent article including, e.g., tissues, sanitary napkins, incontinence devices, diapers, wound care articles including, e.g., dressings, bandages, nursing pads, wrist bands, ankle bands, neck bands, wet wipes, absorbent disposable drapes, meat pad, tissues, toweling, and floor mops.

What is claimed is:

1. A panty liner comprising:
a. a liquid pervious top sheet;
b. a first interior component;
c. a liquid impervious back sheet; and
d. a hot melt superabsorbent polymer composition disposed between the top sheet and the back sheet,
the panty liner having a thickness no greater than 1 millimeter and exhibiting at least one of a 0.9% saline wet back no greater than 30% and a synthetic blood wet back of no greater than 40%.

2. The panty liner of claim 1, wherein the panty liner has a thickness no greater than 0.7 millimeter.

3. The panty liner of claim 1, wherein the panty liner has a thickness no greater than 0.5 millimeter.

4. The panty liner of claim 1, wherein the panty liner has a thickness no greater than 0.3 millimeter

5. The panty liner of claim 1, wherein the thermoplastic superabsorbent polymer composition is adhered to at least one of the top sheet and the back sheet.

6. The panty liner of claim 1, wherein the panty liner exhibits a 0.9% saline wet back of no greater than 20%.

7. The panty liner of claim 1, wherein the panty liner exhibits a 0.9% saline wet back of no greater than 10%.

8. The panty liner of claim 1, wherein the panty liner exhibits a synthetic blood wet back of no greater than 35%.

9. The panty liner of claim 1, wherein the panty liner exhibits a 0.9% saline penetration time of no greater than 10 seconds.

10. The panty liner of claim 1, wherein the panty liner exhibits a synthetic blood penetration time of no greater than 25 seconds.

11. The panty liner of claim 1, wherein the first interior component comprises a gel barrier.

12. The panty liner of claim 1, wherein the first interior component comprises tissue.

13. The panty liner of claim 1, wherein the top sheet comprises a biodegradable nonwoven web, the back sheet comprises a biodegradable polymer film, and the first interior component comprises cellulose tissue.

14. The panty liner of claim 1, wherein the hot melt superabsorbent polymer composition is present in the panty liner in an amount no greater than 0.5 gram.

15. The panty liner of claim 1, further comprising a positioning adhesive adhered to the exterior surface of the back sheet, and optionally a release liner disposed on the positioning adhesive.

16. A panty liner consisting essentially of
a. a liquid pervious top sheet;
b. a first interior component;
c. a liquid impervious back sheet comprising an exterior surface and an interior surface;
d. a hot melt superabsorbent polymer composition disposed between the top sheet and the interior surface of the back sheet;
e. optionally adhesive composition disposed between the top sheet and the back sheet;
f. optionally a positioning adhesive adhered to the exterior surface of the back sheet; and
g. optionally a release liner.

17. The panty liner of claim 1, wherein the adhesive composition is present, the first component being bonded to the top sheet through the adhesive composition and the hot melt superabsorbent polymer composition being bonded to at least one of the first component and the back sheet through the adhesive composition.

18. A panty liner comprising:
a. a liquid pervious top sheet;
b. a first interior component;
c. a liquid impervious back sheet; and
d. a hot melt superabsorbent polymer composition disposed between the top sheet and the back sheet,
the panty liner having a thickness no greater than 1 millimeter.

19. An absorbent article comprising:
a. a liquid pervious top sheet;
b. a liquid impervious back sheet; and
c. a hot melt superabsorbent polymer composition disposed between the top sheet and the back sheet,
the absorbent article having a thickness no greater than 1 millimeter exhibiting at least one of a 0.9% saline wet back no greater than 30% and a synthetic blood wet back of no greater than 40%.
